(19) United States
(12) Patent Application Publication
(10) Pub. No.: US 2024/0042187 A1
(43) Pub. Date: Feb. 8, 2024

(54) FLUID FLOW DISCONNECT SENSOR AND ALARM SYSTEM

(71) Applicant: CareFusion 303, Inc., San Diego, CA (US)

(72) Inventors: Wesley UNDERWOOD, Anaheim, CA (US); Janice R. PAK, Centennial, CO (US); Gene E. MASON, La Habra Heights, CA (US)

(21) Appl. No.: 18/365,340

(22) Filed: Aug. 4, 2023

Related U.S. Application Data
(60) Provisional application No. 63/395,723, filed on Aug. 5, 2022.

(51) Int. Cl. A61M 39/10

(52) U.S. Cl. CPC A61M 39/10; A61M 2039/1005; A61M 2205/18

(57) ABSTRACT

A fluid flow disconnect sensor and alarm system includes first and second sensor units configured to be removably coupled to a first and second portions of a fluid connector assembly, respectively. The first sensor unit comprises a sensor configured to sense when the second sensor unit is within a threshold distance of the first sensor unit, and comprises a transmitter configured to transmit, to a device remote from the first and second sensor units, an indication regarding whether the first and second portions of the fluid connector assembly are coupled together responsive to the sensing. A computing device receiving the indication determines when the first and second portions of the fluid connector assembly are coupled together, and when the assembly becomes disconnected, and may generate an alarm when the first and second portions become disconnected.

### CROSS REFERENCE TO RELATED APPLICATION

This application claims the benefit of priority under 35 U.S.C. § 119 to U.S. Provisional Patent Application Serial No. 63/395,723, entitled “FLUID FLOW DISCONNECT SENSOR AND ALARM SYSTEM”, filed on Aug. 5, 2022, the entire contents of which is incorporated herein by reference.

### TECHNICAL FIELD

The present disclosure is generally related to a control device configured facilitate operation of accessories associated with an infusion device.

### BACKGROUND

During an infusion therapy, a fluid is administered via an administration set connected to a catheter, which may be centrally installed in a large vein. The catheter may be connected to the administration set by a luer. If the administration set is pulled with adequate force, either intentional or accidental, the catheter may dislodge from the patient. Dislodged catheters require replacement with a new catheter, requiring an additional needle stick. It is up to the clinician to notice that a disconnect has occurred when the patient is next checked, which may take up to an hour depending on hospital practice. Upon noticing that the fluid flow has been interrupted, the clinician may clean then rejoin the connection to resume fluid administration. Some administration sets include a fuse, which may block the fluid from flowing on a disconnect. During the time that the fluid path was disconnected, the patient will not receive their medication.

### SUMMARY

According to various aspects, the subject technology provides a system and method for intelligently operating infusion accessory devices. In this regard, an intelligent accessory system is disclosed for use with infusion pumps and related devices. The system increases safety by reducing human error and accessory damage. The system is designed to work with smart accessories that contain a microcontroller and firmware, and which may automatically setup parameters on the pump thereby reducing the setup complexity for the user.

According to various aspects, the subject technology provides a fluid flow disconnect alarm system comprising a first sensor unit configured to be removably coupled to a first portion of a fluid connector assembly, and a second sensor unit configured to be removably coupled to a second portion of a fluid connector assembly and configured to be detected by the first sensor unit, wherein the first sensor unit comprises a sensor configured to sense when the second sensor unit is within a threshold distance of the first sensor unit, and comprises a transmitter configured to transmit, to a device remote from the first and second sensor units, an indication regarding whether the first and second portions of the fluid connector assembly are coupled together responsive to the sensing, and wherein the first and second sensor units are configured such that the threshold distance is satisfied when the first and second portions of the fluid connector assembly are coupled together to form the fluid connector assembly and the threshold distance is not satisfied when the first and second portions are not coupled together. Other aspects include corresponding methods, apparatuses and computer program products for implementation of the corresponding system and its features.

According to some implementations, a disconnect sensor comprises a first sensor unit configured to be removably coupled to a first portion of a fluid connector assembly; and a second sensor unit configured to be removably coupled to a second portion of the fluid connector assembly and configured to be detected by the first sensor unit, wherein the first sensor unit comprises a sensor configured to sense when the second sensor unit is within a threshold distance of the first sensor unit, and comprises a transmitter configured to transmit, to a device remote from the disconnect sensor, an indication regarding whether the first and second portions of the fluid connector assembly are coupled together responsive to the sensing; and wherein the first and second sensor units are configured such that the threshold distance is satisfied when the first and second portions of the fluid connector assembly are coupled together to form the fluid connector assembly and the threshold distance is not satisfied when the first and second portions are not coupled together. Other aspects include corresponding systems, methods, and computer program products for implementation of the foregoing features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1 depicts an example of an institutional patient care system of a healthcare organization, according to aspects of the subject technology.

FIGS. 2A and 2B are conceptual diagrams illustrating an example modular fluid connector assembly, according to aspects of the subject technology.

FIGS. 3A and 3B are conceptual diagrams illustrating an example fluid flow disconnect sensor system, according to aspects of the subject technology.

FIGS. 4A and 4B depict an example a fluid flow disconnect sensor system in use with an example modular fluid connector system, according to aspects of the subject technology.

FIG. 5 depicts an example clinician station, including a computing device for use with the disclosed disconnect sensor, according to aspects of the subject technology.

FIGS. 6A and 6B depict first and second example implementations of a sensor activation mechanism for the disclosed disconnect sensor system, according to various aspects of the subject technology.

FIGS. 7A and 7B depict an example in which sensor activation occurs during coupling of the first and second portions of the fluid connector assembly.

FIGS. 8A and 8B depict a third example implementation of a sensor activation mechanism for the disclosed disconnect sensor, according to various aspects of the subject technology.

FIG. 9 depicts an example process for operating a disconnect sensor and alarm system, according to aspects of the subject technology.

FIG. 10 is a conceptual diagram illustrating an example electronic system for a disconnect sensor and alarm system, according to aspects of the subject technology.

### DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

The subject technology includes an accessory or additional product that may be used along with a fluid connector assembly, or fuse connector, to IV set disconnect events. The subject technology provides disconnect alarm that alerts a clinician when a disconnect has occurred in the IV line, particularly when a fuse-type connector assembly is used. The alert may be provided wirelessly and/or provided via a server in a hospital network to the clinician (e.g., via text message or email, or notification on a designated clinician station). Upon receiving the alert, the clinician may immediately respond to the patient and rejoin the two halves of the connector. The disclosed disconnect sensor is intended to minimize the time that a patient's medication is halted by making the clinician aware that fluid administration is interrupted sooner than what could be detected by intermittent patient status checks.

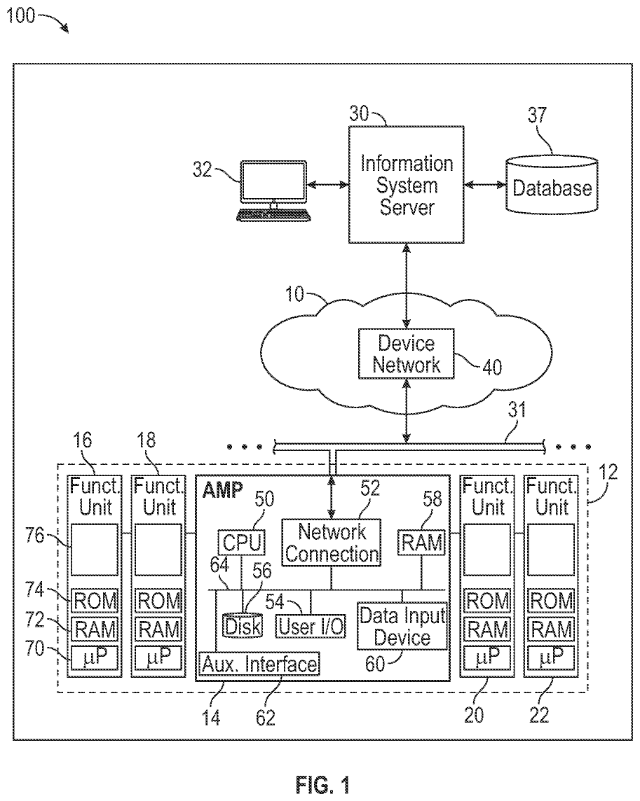

FIG. 1 depicts an example of an institutional patient care system 100 of a healthcare organization, according to aspects of the subject technology. In FIG. 1, a patient care device (or “medical device” generally) 12 is connected to a hospital network 10. The term patient care device (or “PCD”) may be used interchangeably with the term patient care unit (or “PCU”), either which may include various ancillary medical devices such as an infusion pump, a vital signs monitor, a medication dispensing device (e.g., cabinet, tote), a medication preparation device, an automated dispensing device, a module coupled with one of the aforementioned (e.g., a syringe pump module configured to attach to an infusion pump), or other similar devices. Each element 12 is connected to an internal healthcare network 10 by a transmission channel 31. Transmission channel 31 is any wired or wireless transmission channel, for example an 802.11 wireless local area network (LAN). In some implementations, network 10 also includes computer systems located in various departments throughout a hospital. For example, network 10 of FIG. 1 optionally includes computer systems associated with an admissions department, a billing department, a biomedical engineering department, a clinical laboratory, a central supply department, one or more unit station computers and/or a medical decision support system. As described further below, network 10 may include discrete subnetworks. In the depicted example, network 10 includes a device network 40 by which patient care devices 12 (and other devices) communicate in accordance with normal operations.

Additionally, institutional patient care system 100 may incorporate a separate information system server 30, the function of which will be described in more detail below. Moreover, although the information system server 30 is shown as a separate server, the functions and programming of the information system server 30 may be incorporated into another computer, if such is desired by engineers designing the institution's information system. Institutional patient care system 100 may further include one or multiple device terminals 32 for connecting and communicating with information system server 30. Device terminals 32 may include personal computers, personal data assistants, mobile devices such as laptops, tablet computers, augmented reality devices, or smartphones, configured with software for communications with information system server 30 via network 10.

Patient care device 12 comprises a system for providing patient care, such as that described in U.S. Pat. No. 5,713,856 to Eggers et al., which is incorporated herein by reference for that purpose. Patient care device 12 may include or incorporate pumps, physiological monitors (e.g., heart rate, blood pressure, ECG, EEG, pulse oximeter, and other patient monitors), therapy devices, and other drug delivery devices may be utilized according to the teachings set forth herein. In the depicted example, patient care device 12 comprises a interface device 14, also referred to as interface unit 14, connected to one or more functional modules 16, 18, 20, 22. Interface unit 14 includes a central processing unit (CPU) 50 connected to a memory, for example, random access memory (RAM) 58, and one or more interface devices such as user interface device 54, a coded data input device 60, a network connection 52, and an auxiliary interface 62 for communicating with additional modules or devices. Interface unit 14 also, although not necessarily, includes a main non-volatile storage unit 56, such as a hard disk drive or non-volatile flash memory, for storing software and data and one or more internal buses 64 for interconnecting the aforementioned elements.

In various implementations, user interface device 54 is a touch screen for displaying information to a user and allowing a user to input information by touching defined areas of the screen. Additionally, or in the alternative, user interface device 54 could include any means for displaying and inputting information, such as a monitor, a printer, a keyboard, softkeys, a mouse, a track ball and/or a light pen. Data input device 60 may be a bar code reader capable of scanning and interpreting data printed in bar coded format. Additionally, or in the alternative, data input device 60 can be any device for entering coded data into a computer, such as a device(s) for reading a magnetic strip, radio-frequency identification (RFID) devices whereby digital data encoded in RFID tags or smart labels (defined below) are captured by the reader 60 via radio waves, PCMCIA smart cards, radio frequency cards, memory sticks, CDs, DVDs, or any other analog or digital storage media. Other examples of data input device 60 include a voice activation or recognition device or a portable personal data assistant (PDA). Depending upon the types of interface devices used, user interface device 54 and data input device 60 may be the same device. Although data input device 60 is shown in FIG. 1 to be disposed within interface unit 14, it is recognized that data input device 60 may be integral within pharmacy system 34 or located externally and communicating with pharmacy system 34 through an RS-232 serial interface or any other appropriate communication means. Auxiliary interface 62 may be an RS-232 communications interface, however any other means for communicating with a peripheral device such as a printer, patient monitor, infusion pump or other medical device may be used without departing from the subject technology. Additionally, data input device 60 may be a separate functional module, such as modules 16, 18, 20 and 22, and configured to communicate with controller 14, or any other system on the network, using suitable programming and communication protocols.

Network connection 52 may be a wired or wireless connection, such as by Ethernet, WiFi, BLUETOOTH, an integrated services digital network (ISDN) connection, a digital subscriber line (DSL) modem, or a cable modem. Any direct or indirect network connection may be used, including, but not limited to a telephone modem, an MIB system, an RS232 interface, an auxiliary interface, an optical link, an infrared link, a radio frequency link, a microwave link, a personal area network connection, a local area network connection, a cellular link, or a WLANS connection or other wireless connection.

Functional modules 16, 18, 20, 22 are any devices for providing care to a patient or for monitoring patient condition. As shown in FIG. 1, at least one of functional modules 16, 18, 20, 22 may be an infusion pump module such as an intravenous infusion pump for delivering medication or other fluid to a patient. For the purposes of this discussion, functional module 16 is an infusion pump module. Each of functional modules 18, 20, 22 may be any patient treatment or monitoring device including, but not limited to, an infusion pump, a syringe pump, a patient controlled analgesia (PCA) pump, an epidural pump, an enteral pump, a blood pressure monitor, a pulse oximeter, an EKG monitor, an EEG monitor, a heart rate monitor, or an intracranial pressure monitor or the like. Functional module 18, 20 and/or 22 may be a printer, scanner, bar code reader or any other peripheral input, output or input/output device.

Each functional module 16, 18, 20, 22 communicates directly or indirectly with interface unit 14, with interface unit 14 providing overall monitoring and control of device 12. Functional modules 16, 18, 20, 22 may be connected physically and electronically in serial fashion to one or both ends of interface unit 14 as shown in FIG. 1, or as detailed in Eggers et al. However, it is recognized that there are other means for connecting functional modules with the interface unit that may be utilized without departing from the subject technology. It will also be appreciated that devices such as pumps or patient monitoring devices that provide sufficient programmability and connectivity may be capable of operating as stand-alone devices and may communicate directly with the network without connected through a separate interface unit or control unit 14. As described above, additional medical devices or peripheral devices may be connected to patient care device 12 through one or more auxiliary interfaces 62.

Each functional module 16, 18, 20, 22 may include module-specific components 76, a microprocessor 70, a volatile memory 72 and a nonvolatile memory 74 for storing information. It should be noted that while four functional modules are shown in FIG. 1, any number of devices may be connected directly or indirectly to central controller 14. The number and type of functional modules described herein are intended to be illustrative, and in no way limit the scope of the subject technology. Module-specific components 76 include any components necessary for operation of a particular module, such as a pumping mechanism for infusion pump module 16.

While each functional module may be capable of a least some level of independent operation, interface unit 14 monitors and controls overall operation of device 12. For example, as will be described in more detail below, interface unit 14 provides programming instructions to the functional modules 16, 18, 20, 22 and monitors the status of each module.

Patient care device 12 is capable of operating in several different modes, or personalities, with each personality defined by a configuration database. The configuration database may be a database 56 internal to patient care device, or an external database 37. A particular configuration database is selected based, at least in part, by patient-specific information such as patient location, age, physical characteristics, or medical characteristics. Medical characteristics include, but are not limited to, patient diagnosis, treatment prescription, medical history, medical records, patient care provider identification, physiological characteristics or psychological characteristics. As used herein, patient-specific information also includes care provider information (e.g., physician identification) or a patient care device's 10 location in the hospital or hospital computer network. Patient care information may be entered through interface device 52, 54, 60 or 62, and may originate from anywhere in network 10, such as, for example, from a pharmacy server, admissions server, laboratory server, and the like.

Medical devices incorporating aspects of the subject technology may be equipped with a network interface module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, patient care device 12 and network 10 may communicate via automated interaction, manual interaction, or a combination of both automated and manual interaction. Automated interaction may be continuous or intermittent and may occur through direct network connection 54 (as shown in FIG. 1), or through RS232 links, MIB systems, RF links such as BLUETOOTH, IR links, PANS, LANS, WLANS, digital cable systems, telephone modems or other wired or wireless communication means. Manual interaction between patient care device 12 and network 10 involves physically transferring, intermittently or periodically, data between systems using, for example, user interface device 54, coded data input device 60, bar codes, computer disks, portable data assistants, memory cards, or any other media for storing data. The communication means in various aspects is bidirectional with access to data from as many points of the distributed data sources as possible. Decision-making can occur at a variety of places within network 10. For example, and not by way of limitation, decisions can be made in HIS server 30, decision support 48, remote data server 49, hospital department or unit stations 46, or within patient care device 12 itself.

All direct communications with medical devices operating on a network in accordance with the subject technology may be performed through information system server 30, known as the remote data server (RDS). In accordance with aspects of the subject technology, network interface modules incorporated into medical devices such as, for example, infusion pumps or vital signs measurement devices, ignore all network traffic that does not originate from an authenticated RDS. The primary responsibilities of the RDS of the subject technology are to track the location and status of all networked medical devices that have NIMs, and maintain open communication

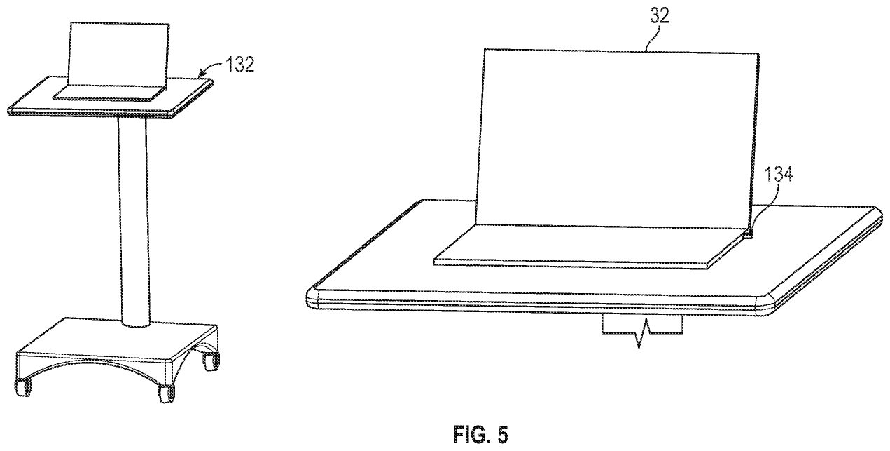

In some implementations, medication delivery modules 16, 18, 20, 22 include plug-in ports for expansion. Accordingly, a new medication delivery module may be attached to PCU 12 by coupling a connector through the plug-in ports, which may include electrical terminals so that the added medication delivery module 16, 18, 20, 22 may transmit and receive information to and from a control module 14. In some implementations, the added medication delivery module 16, 18, 20, 22 may also receive power from control module 14 through a plug-in port. Control module 14 may include a main display, a memory and a processor (see FIG. 5), and may be configured to display operational parameters and medication delivery status, and further information associated with each of medication delivery modules 16, 18, 20, 22. According to various implementations, module displays may also display physiological data (e.g., vital signs) associated with a patient.

A main display (e.g., I/O 54) may be configured to display one or more user interfaces for the display of operational parameters or other data associated with a module 16, 18, 20, 22, and/or physiological parameters associated with the patient. The main display may include multiple user interfaces, with each individual user interface graphically displaying information for a respective one of medication modules, including information also displayed on a corresponding module displays. In some implementations, control module 14 includes a communications module (including, e.g., an antenna), configured to communicate wirelessly with a controller, or with a network.

With reference to FIG. 1, when a medication delivery module 16, 18, 20, 22 initiates an infusion of a medication to the patient, the control module 14 is configured to create and manage an infusion session within a memory of the control module (or related module). For the purpose of this disclosure, the infusion session includes state information of the PCU 12, its control module 14, and/or its associated modules, which is recorded and saved to memory during a particular period of time. The state information includes, but is not limited to, records of parameter values utilized by the PCU, its control module, and/or its associated modules during the period of time, and/or records physiological data collected during the period of time. During the infusion, physiological data associated with the patient is recorded within the session, operating parameter values, and any modifications to the operating parameters of the PCU, its control module, and/or modules are also recorded in the session.

If not already logged into the PCU 12, the clinician may scan his or her badge proximate to a sensor (e.g., 54, 60) on the PCU 12, and the PCU may attempt to authenticate the clinician by sending the clinician's scanned identification to server 30. The clinician's badge may incorporate a radio frequency identification device (RFID), which is read by a scanner integrated with the PCU, or a portable scanner associated with the PCU. The clinician may scan his or her badge at the control module 14 to identify and authorize the clinician to initiate the administration of a medication. Once the clinician is associated with the PCU and/or module(s), the clinician's identification is associated with the session. The same is applicable with a patient. The clinician may scan the patient's wristband with a portable scanner, or using the sensor on the PCU 12 (or its control module) to associate the patient with the PCU and/or module(s) (and a session).

The control unit 14 of PCU 12 is configured to generate a graphical representation of the infusion session, and display (e.g., in a display) the graphical representation, including a graphical visualization of all parameters of the infusion during the session and any modifications any modifications to the parameters, together with physiological data obtained during the session. The graphical representation may include pseudo identifiers for unknown data until such data is substituted with known identifiers.

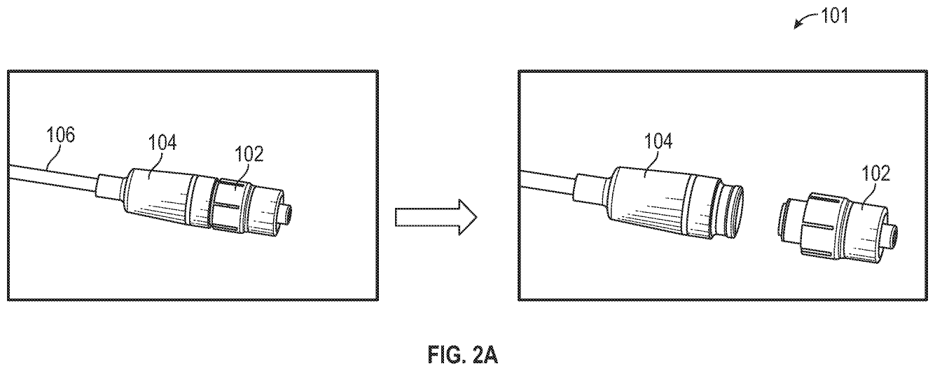

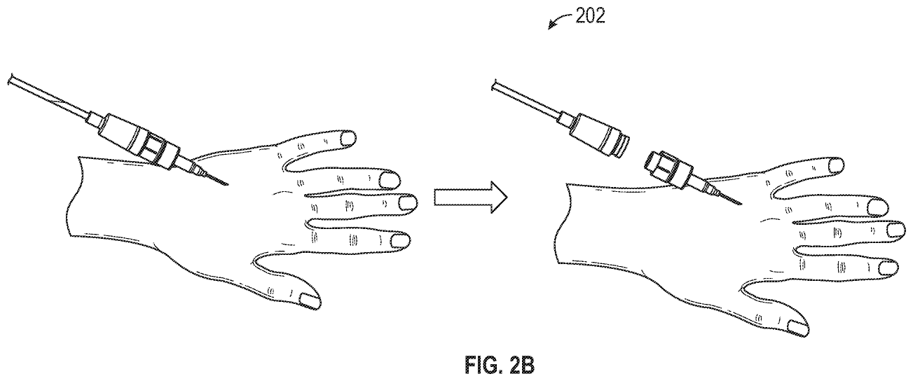

FIGS. 2A and 2B are conceptual diagrams illustrating an example modular fluid connector assembly 101, according to aspects of the subject technology. The fluid connector assembly 101 may be directed to any modular fluid delivery system having two or more parts 102, 104 that may be connected together to form a fluid channel. For example, the fluid connector assembly may include a first component configured to connect to an intravenous (IV) tubing, and a second component including a luer for connection to a catheter or other subcutaneous insertion device. The depicted system includes first and second portions 102, 104 of a fuse-type connector. The first portion 102 of the fluid connector assembly 101 includes a first portion of a valve (not shown) and a first fluid passageway therein. The second portion of the fluid connector assembly 101 includes a second portion of the valve and a second fluid passageway therein. According to some implementations, the second assembly is configured to become coupled to the first assembly to form a continuous fluid passage with a valve, and the functions as a fuse to halt fluid flow when it becomes disconnected. When the two components are connected, fluid flow is open from end to end; for example, fluid may flow from the IV tubing to the patient. If a force is applied to the device and/or line, the device components may separate and stop fluid flow through the device. An example assembly for implementation of the subject technology is described in U.S. Application No. 63/217,165, filed Jun. 30, 2021, incorporated herein in its entirety.

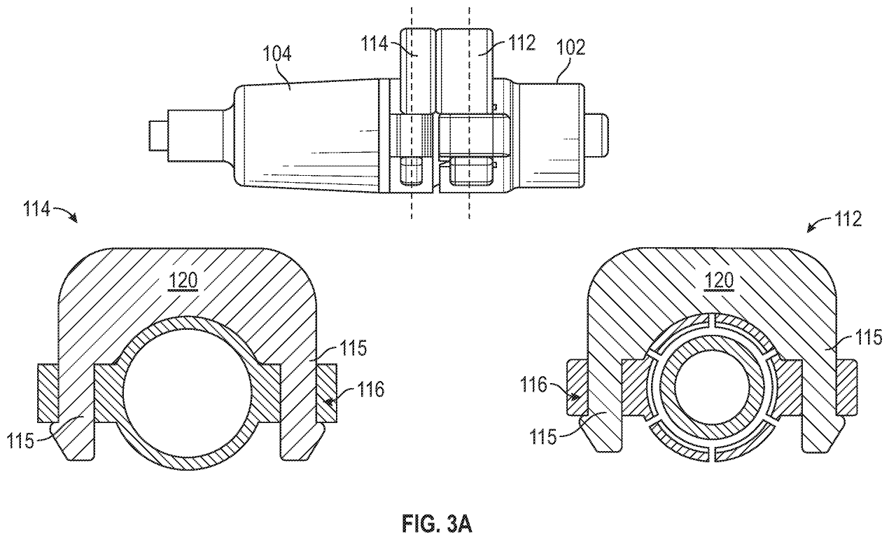

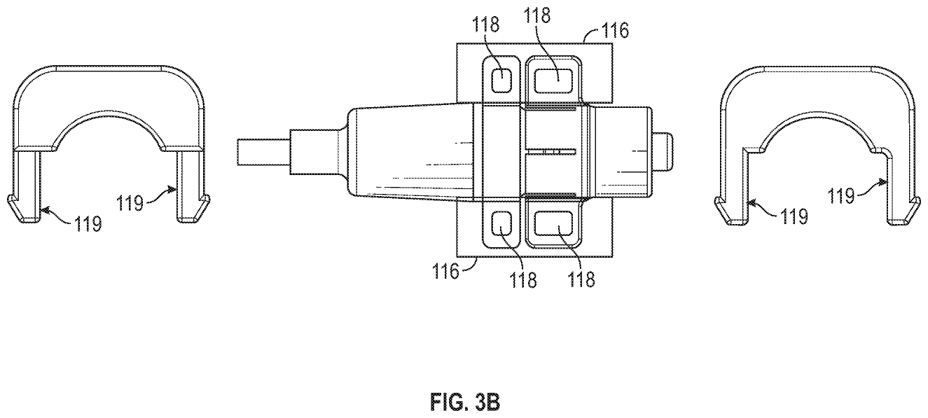

FIGS. 3A and 3B are conceptual diagrams illustrating an example fluid flow disconnect sensor system, according to aspects of the subject technology. According to various implementations, the fluid flow disconnect sensor system includes a first sensor unit 112 configured to be removably coupled to a first portion 102 of a fluid connector assembly, and a second sensor unit 114 configured to be removably coupled to a second portion 104 of the fluid connector assembly 101. According to various implementations, the second sensor unit 114 is configured to be detected by the first sensor unit 112. In some implementations, the first sensor unit may be configured to be detected by the second sensor unit.

As will be described further, the first sensor unit 112 includes a sensor configured to sense when the second sensor unit 114 is within a threshold distance of the first sensor unit 112.

The sensor may be housed within the body of the sensor unit 112. For the purpose of this disclosure, a “threshold distance” is used to impart or describe a configuration of the two sensor units and may not describe a particular distance. For example, the threshold distance may include a distance inferred from the configuration, and/or that may be a result or consequence of its implementation with the fluid connector assembly 101. The first sensor unit 112 (or second sensor unit 114) includes a transmitter configured to transmit, to a device remote from the units, an indication regarding whether the first and second portions 102, 104 of the fluid connector assembly 101 are coupled together responsive to the sensing.

Each of the sensor units 112, 114 may be oblong or square shaped, with a pair of parallel locking appendages 115 extending from its sides, as depicted by FIGS. 3A and 3B. Also as depicted, each portion 102, 104 of the fluid connector assembly 101 may be configured with one or more interfacing tabs 116 configured to receive the locking appendages. The interfacing tabs 116 may be formed as part of the component portion's body, or may be detachably connected to the body of the component, for example, by snapping around the body. Each tab 116 may include an aperture (or key opening) 118. The locking appendages may include interfacing teeth 119 (e.g., each with a one directional ridge) which may be received into apertures 118 and lock the sensing unit in place. In this regard, the first sensor unit 112 may be configured to be removably coupled to the first portion of the fluid connector assembly 102 via one or more first tabs 116a secured to the exterior the first portion 102 of the fluid connector assembly 101, and the second sensor unit 114 may be configured to be removably coupled to the second portion 104 of the fluid connector assembly 101 via one or more second tabs 116b secured to an exterior of the second portion 104.

According to various implementations, when a respective sensor unit 112, 114 is coupled to a respective portion 102, 104 of fluid connector assembly 101, the appendages straddle the connector assembly on opposing sides of the connector assembly and lock into respective key openings 118 within the tabs 116 of the assembly 102, 104 on the opposing sides so that the respective sensor unit 112, 114 is held against a side of the fluid connector assembly, as depicted in FIG. 3A.

As depicted, the body (or housing) 120 of each sensing unit 112, 114 may form a partial rectangle with one side bored out to form around a connector assembly. An upper, majority portion 120 of each sensor unit may have at least half of a mass of the entire sensor unit. The lateral sides (e.g., perpendicular to the fluid path) of majority portion may extend into the appendages such that the lateral sides and appendages for a side of the partial rectangle, and a top of the majority portion forms another side. What would be a fourth side is bored out to form around the connector assembly, as shown. When installed, the sensor unit is held by the locking appendages in a position perpendicular to the side of the connector assembly between the tabs, as shown in FIG. 3A. The longitudinal sides (e.g., in the direction of the fluid path) are substantially flat. In some implementations, each sensor unit 112, 114 has at least one flat side facing a flat side of the other sensor unit when the sensor units are held in place by the locking appendages.

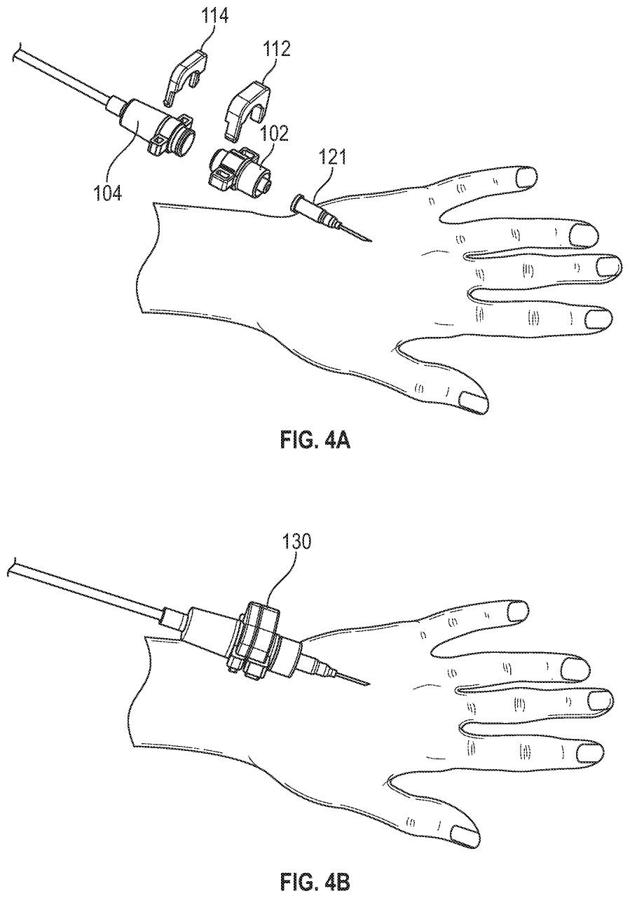

FIGS. 4A and 4B depict an example a fluid flow disconnect sensor system in use with an example modular fluid connector system, according to aspects of the subject technology. FIG. 4A depicts an exploded illustration. As depicted, a fluid connector assembly 101 may include a first fuse portion 102 tube end, that may be configured to prime with a fluid from a IV tubing 106 (from an infusion pump 12), and a second fuse portion 104 that may be configured as a luer end for connecting to a catheter 122. The sensor units 112, 114 are depicted above the fluid connector assembly 101, ready for insertion/docking to respective tabs 116 of the fluid connector system 101. FIG. 4B depicts the complete assembled system 130.

FIG. 5 depicts an example clinician station 132, including a computing device for use with the disclosed disconnect sensor, according to aspects of the subject technology. According to various aspects of the subject technology, the transmitter of sensor unit 112 may be configured to wirelessly connect to a remote computing device 32. The transmitter may include short-range wireless communication circuity for pairing to and exchanging data with corresponding receivers. For example, the sensor unit 112 may communicate using Bluetooth. In such cases, the sensor unit 112 may be paired with a Bluetooth compatible device, such as a laptop, desktop, or mobile device. In some implementations, the sensor unit may communicate using radio-frequency (RF) transmission. In some implementations, a dongle 134 may be utilized at the computing device to provide receiving and transmitting functionality to the computing device 32. In some implementations, particularly in situations where wireless communications may interfere with other systems, a wired technology may be utilized. For example, an electronic wire/tether may extend between the dongle 134 and the sensor unit 112, providing communications between the sensor unit and the computing device.

According to various implementations, a non-transitory computer readable medium may be provided that includes software for execution by the computing device 32 to facilitate communications with the disconnect sensor and interoperability with other medical systems, such as infusion pumps 12 and med stations. The software may be loaded on a USB stick associated with or integrated into the disconnect sensor (e.g., in first sensor 112). The software may be compatible with an operating system of the terminal computer 32.

With brief reference to FIGS. 3A and 10, the disconnect sensor system may include a microprocessor and/or memory device. In this regard, the memory device may be used to store information such as a patient identifier. A scanning device (not shown) may be integrated with or connected to the computing device. Software may be initiated at the computing device by scanning or inputting an identifier of the disconnect sensor, in connection with the setup of a infusion pump 12 to administer medication to a patient. The clinician may start the pump setup workflow (e.g., via input device 54, 60 or 62 on PCU 12 and/or via terminal 32), scan an identifier on the sensor unit (e.g., a barcode, QR code, or an RFID tag in the second sensor unit 114), scan an identifier on the fluid connector assembly 101, enter a patient identifier, connect the IV set (e.g., IV tubing catheter, etc.) and initiate the infusion. In some implementations, a barcode scanner may be integrated with or connected to PCU 14 (e.g., input device 54, 60 or 62 on PCU, 12).

According to some implementations, the software operating on the computing device associates the patient identifier with the first and second sensor units 112, 114, and stores the patient identifier in a memory device (e.g., in local memory or a database 37 associated with a server 30). During operation, indications are sent from the sensor unit 112 to the computing device (e.g., over a wireless connection) indicating whether the disconnect sensor has sensed a disconnection; e.g., whether the second sensor unit is within a threshold distance of the first sensor unit. The patient identifier stored locally by the disconnect sensor 101 is sent in connection with the disconnect status changing (e.g., becoming connected or disconnected). In this regard, the computing device may monitor (e.g., via the dongle 134) whether the patient identifier has changed by way of connecting a respective portion of the fluid connector device to a different patient. On a change in connection status, the computing device confirms that the received patient identifier is associated with the first and second sensor unit 112, 114. If the computing device detects a change then an alert may be provided audibly or visually on the display screen. On detecting a change, or periodically, the computing device may provide the coupling status of the first and second portions of the fluid connector assembly and whether the obtained patient identifier is associated with the first and second sensor units. In some implementations, the sensor may not send a disconnection alert signal to the dongle 134 until a disconnection is detected.

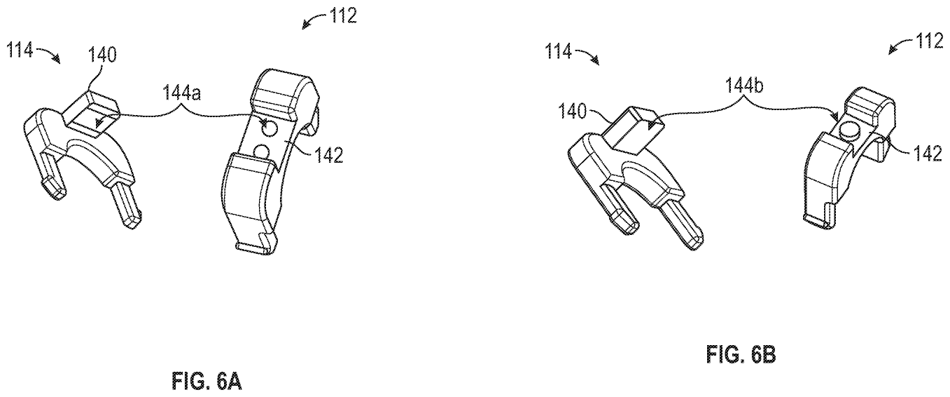

FIGS. 6A and 6B depict first and second example implementations of a sensor activation mechanism for the disclosed disconnect sensor system, according to various aspects of the subject technology. In some implementations, the body (or majority) portion 120 of one of the sensor units includes an overhang portion 140 that extends away from the respective sensor to align with and couple to an interface 142 in the body portion 120 of the other sensor unit when the first and second sensor units 112, 114 are held by the locking appendages 115 and the first and second portions 102, 104 of the fluid connector assembly 101 are coupled together. In some implementations, as depicted in FIGS. 6A and 6B, the interface portion 142 may be in the form of a notch into which the overhang portion 140 of the other unit may slide into. In this manner, the sensor units are forced to be aligned when joined together. In the depicted example, the second sensor unit 114 includes the overhang; however, in some implementations, the first sensor unit 112 may include the overhang.

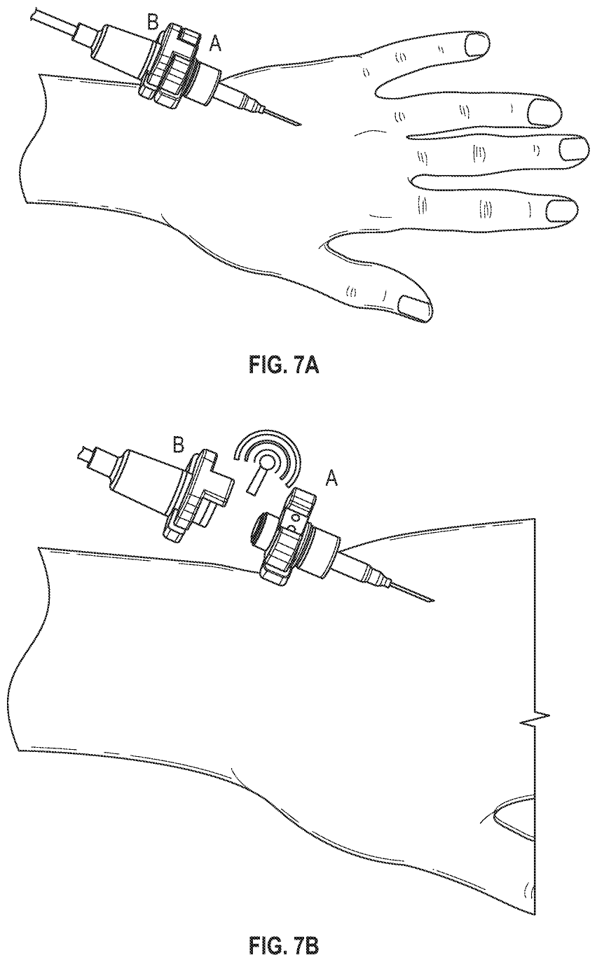

FIGS. 7A and 7B depict an example in which sensor activation occurs during coupling of the first and second portions 102, 104 of the fluid connector assembly. With reference to FIGS. 6 and 7, according to various implementations, the interface or the overhang portion comprises a trigger contact 144 that is activated responsive to the overhang portion 140 aligning with and coupling to the interface 142. In the example of FIG. 6A, the trigger contact is a contact for completing an electrical circuit. In this regard, the trigger contact 144a includes two electrical conducting pads that, when shorted, provides a current within the device that is detected by the sensor, which reads the short as the two portions being coupled. In this manner, the disconnect sensor may determine that the portions 112, 114 are not coupled when the pads are not shorted. The overhang portion includes an electrical conducting pad underneath the overhang which, when the two portions are joined, overlaps and makes contact with the electrical pads 144a, causing the short.

In the example, of FIG. 6B, the trigger contact 144b includes a push button. In the depicted implementation, the push button is activated by the overhand as the overhang 140 slides into the interface 142 where the push button is located. The overhang 142 slides into the interface 140 and, by way of the sensor units being anchored into the respective portions of the fluid connector assembly 101, the overhang presses down on the button 144b, causing the disconnect sensor to detect that the portions are coupled. The disconnect sensor may determine that the portions are not coupled when the button is not depressed.

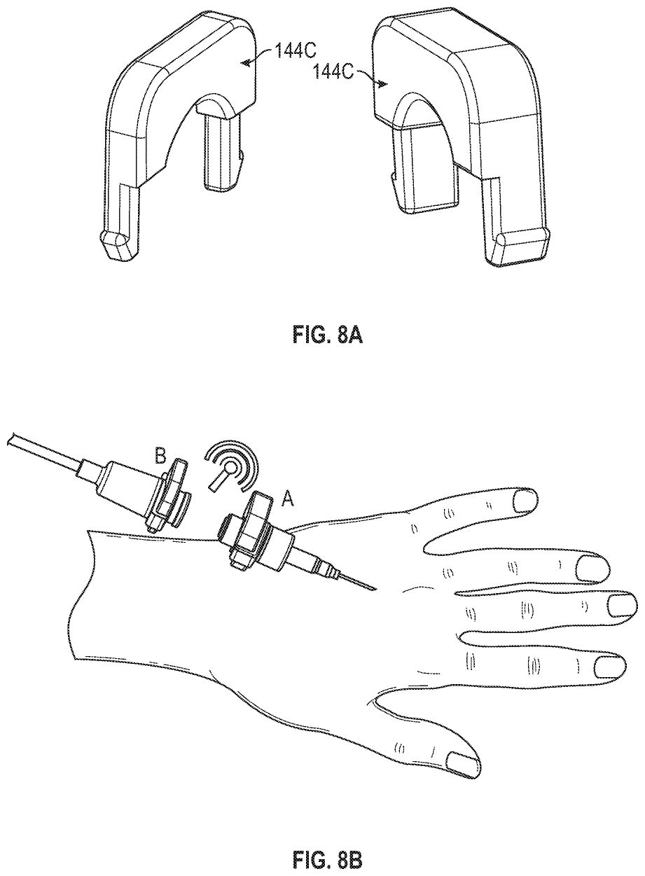

FIGS. 8A and 8B depict a third example implementation of a sensor activation mechanism for the disclosed disconnect sensor, according to various aspects of the subject technology. In the depicted implementation, the trigger contact 144c includes one of the sensor units incorporating an electronic tag (e.g., an RFID chip), and the other sensor unit incorporating a sensor configured to detect the electronic tag while the first and second sensor units are brought together by way of the first and second portions of the fluid connector assembly being coupled together to form the fluid connector assembly 101. In depicted example, the second sensor unit 114 includes the electronic tag, while the first sensor unit 112 includes a sensor for detecting the tag within sensor 114.

In some implementations (e.g., in FIG. 8B), the sensor is configured to detect the presence of the tag when it is sufficiently close to the sensor such that the first and second portions 102, 104 of the fluid connector assembly 101 are connected (and the sensor units are abutting each other or sufficiently close to each other). Likewise, the sensor is configured to no longer detect the presence of the electronic tag when the first and second sensor units are moved away from each other by way of the first and second portions of the fluid connector assembly being decoupled. As described previously, the sensor unit responsible for sensing the tag may be configured to transmit a coupling status indication when the presence of the electronic tag is detected or no longer detected. It's noted that the overhang portion 140 is used in the depicted implementation; however, an overhang portion 140 and corresponding interface 142 may be included in the respective sensor units 112, 114 for proper alignment of the units, if desired.

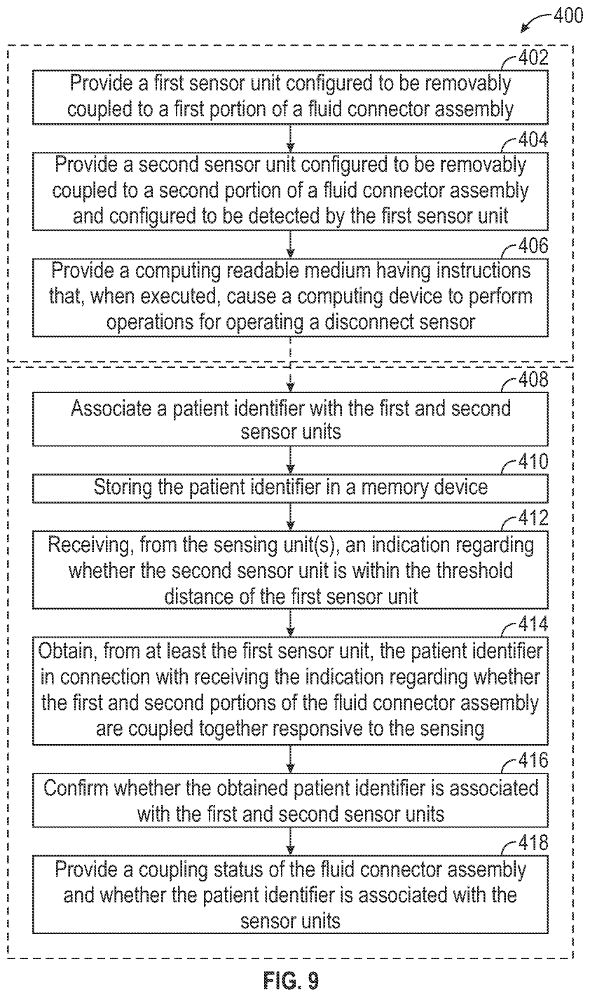

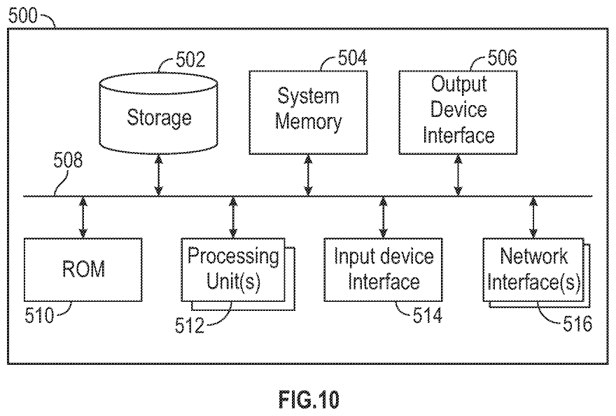

FIG. 9 depicts an example process for operating a disconnect sensor and alarm system, according to aspects of the subject technology. For explanatory purposes, the various blocks of example process 900 are described herein with reference to FIGS. 1 through 8, and the components and/or processes described herein. For explanatory purposes, the blocks of example process 900 are described as occurring in serial, or linearly. However, multiple blocks of example process 900 may occur in parallel. In addition, the blocks of example process 900 need not be performed in the order shown and/or one or more of the blocks of example process 900 need not be performed.

In the depicted example, a first sensor unit configured to be removably coupled to a first portion of a fluid connector assembly is provided (402). A second sensor unit configured to be removably coupled to a second portion of a fluid connector assembly and configured to be detected by the first sensor unit, is also provided (404). As described previously, the first sensor unit may include a sensor configured to sense when the second sensor unit is within a threshold distance of the first sensor unit, and may include a transmitter configured to transmit, to a device remote from the first and second sensor units, an indication regarding whether the first and second portions of the fluid connector assembly are coupled together responsive to the sensing. The first and second sensor units may be configured such that the threshold distance is satisfied when the first and second portions of the fluid connector assembly are coupled together to form the fluid connector assembly and the threshold distance is not satisfied when the first and second portions are not coupled together.

According to some implementations, at least the second sensor unit comprises a memory device for storing a patient identifier. A computer readable medium is further provided with the devices (406). The computer readable medium has instructions stored thereon that, when executed by a computing device, cause the computing device to perform operations for operating a disconnect sensor. The operations include steps that may be included in process 400, or may be the subject of a separate process.

According to various implementations, the operations performed by the instructions include associating, at the computing device, a patient identifier with the first and second sensor units (408), storing the patient identifier in the memory device (410), receiving an indication regarding whether the second sensor unit is within the threshold distance of the first sensor unit (412), and obtaining, from at least the first sensor unit, the patient identifier in connection with receiving the indication regarding whether the first and second portions of the fluid connector assembly are coupled together responsive to the sensing (414). The process may further include confirming whether the obtained patient identifier is associated with the first and second sensor units (416) and providing, for display based on the obtaining and confirming, a coupling status of the first and second portions of the fluid connector assembly and whether the obtained patient identifier is associated with the first and second sensor units (418).

According to various implementations, a clinician's workflow begins by ensuring that the dongle 134 and the software are installed on the clinician workstation 32. The clinician installs the disconnect sensor (sensor and tag portions 112, 114) on a fluid connector assembly 101. The clinician may then proceed to register the patient identifier (e.g., patient identification number or room number) within the software and links the identifier with the disconnect sensor. If the fluid connector assembly 101 is disconnected, the sensor portion 112 of the disconnect sensor detects that the tag portion 114 is out of contact and sends an alert signal to the dongle 134. The clinician may then reconnect the connector assembly, restoring contact between the first and second portions 112, 114 of the disconnect sensor, and the medication administration continues.

In some implementations, the first and/or second sensor unit 112, 114 may include a piezo-electric circuit configured to generate an audible alarm. If the fluid connector assembly 101 is disconnected, the piezo-electric circuit may be instructed to generate the alarm. The instruction may be a signal generated within the sensor unit, or may be sent from the dongle 134. In this manner, the audible alarm emanates from the separated connector itself in addition to, or in the alternative of, the previously described remote system alarm.

Many of the above-described example 400, and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

FIG. 10 is a conceptual diagram illustrating an example electronic system 500 for a disconnect sensor and alarm system, according to aspects of the subject technology. Electronic system 500 may be a computing device for execution of software associated with one or more portions or steps of process 500, or components and processes provided by FIGS. 1-9, including but not limited to information system server 30, database 37, computing hardware within patient care device 12, or a remote device 32 (e.g., a mobile device). Electronic system 500 may be representative, in combination with the disclosure regarding FIGS. 1-9. In some implementations, electronic system 500 may be representative of circuitry within one of the sensor units 112, 114; for example, the first sensor unit 112 with the input device interface 514 representative of a sensor capable of detecting the second sensor unit 112 (e.g., a trigger contact 144 or RFID reader). In this regard, electronic system 500 may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 500 may include various types of computer readable media and interfaces for various other types of computer readable media. In the depicted example, electronic system 500 includes a bus 508, processing unit(s) 512, a system memory 504, a read-only memory (ROM) 510, a permanent storage device 502, an input device interface 514, an output device interface 506, and one or more network interfaces 516. In some implementations, electronic system 500 may include or be integrated with other computing devices or circuitry for operation of the various components and processes previously described.

Bus 508 collectively represents all system, peripheral, and chipset buses that communicatively connect the numerous internal devices of electronic system 500. For instance, bus 508 communicatively connects processing unit(s) 512 with ROM 510, system memory 504, and permanent storage device 502.

From these various memory units, processing unit(s) 512 retrieves instructions to execute and data to process, in order to execute the processes of the subject disclosure. The processing unit(s) can be a single processor or a multi-core processor in different implementations.

ROM 510 stores static data and instructions that are needed by processing unit(s) 512 and other modules of the electronic system. Permanent storage device 502, on the other hand, is a read-and-write memory device. This device is a non-volatile memory unit that stores instructions and data even when electronic system 500 is off. Some implementations of the subject disclosure use a mass-storage device (such as a magnetic or optical disk and its corresponding disk drive) as permanent storage device 502.

Other implementations use a removable storage device (such as a floppy disk, flash drive, and its corresponding disk drive) as permanent storage device 502. Like permanent storage device 502, system memory 504 is a read-and-write memory device. However, unlike storage device 502, system memory 504 is a volatile read-and-write memory, such as a random access memory. System memory 504 stores some of the instructions and data that the processor needs at runtime. In some implementations, the processes of the subject disclosure are stored in system memory 504, permanent storage device 502, and/or ROM 510. From these various memory units, processing unit(s) 512 retrieves instructions to execute and data to process in order to execute the processes of some implementations.

Bus 508 also connects to input and output device interfaces 514 and 506. Input device interface 514 enables the user to communicate information and select commands to the electronic system. Input devices used with input device interface 514 include, e.g., alphanumeric keyboards and pointing devices (also called “cursor control devices”). Output device interfaces 506 enables, e.g., the display of images generated by the electronic system 500. Output devices used with output device interface 506 include, e.g., printers and display devices, such as cathode ray tubes (CRT) or liquid crystal displays (LCD). Some implementations include devices such as a touchscreen that functions as both input and output devices.

Also, as shown in FIG. 10, bus 508 also couples electronic system 500 to a network (e.g., network 40 of FIG. 1) through network interfaces 516. Network interfaces 516 may include, e.g., a wireless access point (e.g., Bluetooth or WiFi) or radio circuitry for connecting to a wireless access point. Network interfaces 516 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 500 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

### Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identification

Clause 1. A fluid flow disconnect system comprising: a first sensor unit configured to be removably coupled to a first portion of a fluid connector assembly; and a second sensor unit configured to be removably coupled to a second portion of a fluid connector assembly and configured to be detected by the first sensor unit, wherein the first sensor unit comprises a sensor configured to sense when the second sensor unit is within a threshold distance of the first sensor unit, and comprises a transmitter configured to transmit, to a device remote from the first and second sensor units, an indication regarding whether the first and second portions of the fluid connector assembly are coupled together responsive to the sensing; wherein the first and second sensor units are configured such that the threshold distance is satisfied when the first and second portions of the fluid connector assembly are coupled together to form the fluid connector assembly and the threshold distance is not satisfied when the first and second portions are not coupled together.

Clause 2. The fluid flow disconnect system of Clause 1, wherein the first sensor unit is configured to be removably coupled to the first portion of the fluid connector assembly via one or more first tabs secured to an exterior the first portion of the fluid connector assembly, and the second sensor unit is configured to be removably coupled to the second portion of the fluid connector assembly via one or more second tabs secured to an exterior of the second portion of the fluid connector assembly.

Clause 3. The fluid flow disconnect system of Clause 2, wherein the first and second sensor units each include a pair of parallel locking appendages that, when the respective sensor unit is coupled to a respective portion of the fluid connector assembly, straddle the respective portion of the fluid connector assembly on opposing sides of the respective portion of the fluid connector assembly and lock into respective key openings within the tabs of the respective portion of the fluid connector assembly on the opposing sides so that the sensor unit is held against a side of the respective portion of the fluid connector assembly between the tabs.

Clause 4. The fluid flow disconnect system of Clause 3, wherein, when a respective sensor unit is coupled to a respective portion of the fluid connector assembly, a majority portion of the sensor unit having at least half of a mass of the sensor unit is held by the locking appendages in a position perpendicular to the side of the respective portion of the fluid connector assembly between the tabs, wherein the majority portion of the first sensor unit comprises at least one flat side facing a flat side of the majority portion of the second sensor unit when the sensor units are held by the locking appendages.

Clause 5. The fluid flow disconnect system of Clause 4, wherein the majority portion of a respective sensor unit comprises an overhang portion that extends away from the respective sensor unit to align with and couple to an interface in the majority portion of the other sensor unit when the first and second sensor units are held by the locking appendages and the first and second portions of the fluid connector assembly are coupled together.

Clause 6. The fluid flow disconnect system of Clause 5, wherein interface or the overhang portion comprises a trigger contact that is activated responsive to the overhang portion aligning with and coupling to the interface, and wherein the first sensor unit is configured to detect the second sensor unit when the trigger contact is activated and to detect that the first and second sensor units are disconnected with the trigger contact is deactivated.

Clause 7. The fluid flow disconnect system of Clause 6, wherein the trigger contact comprises a push button or a contact for completing an electrical circuit.

Clause 8. The fluid flow disconnect system of any one of Clauses 1-7, wherein the transmitter is configured to wirelessly transmit the indication to the remote device.

Clause 9. The fluid flow disconnect system of any one of Clauses 1-8, further comprising the first portion of the fluid connector assembly and the second portion of the fluid connector assembly, wherein the first portion of the fluid connector assembly comprises a first portion of a valve and a first fluid passageway therein; and wherein the second portion of the fluid connector assembly comprises a second portion of the valve and a second fluid passageway therein, the second portion of the fluid connector assembly being configured to become coupled to the first portion of the fluid connector assembly to form a continuous fluid passage from the first and second fluid passageways.

Clause 10. The fluid flow disconnect system of any one of Clauses 1-5, 8, and 9, wherein the second sensor unit comprises a electronic tag, and the sensor of the first sensor unit is configured to wirelessly detect a presence of the electronic tag of the second sensor unit while the first and second sensor units are brought together by way of the first and second portions of the fluid connector assembly being coupled together to form the fluid connector assembly, and configured to no longer detect the presence of the electronic tag when the first and second sensor units are moved away from each other by way of the first and second portions of the fluid connector assembly being decoupled, and wherein the first sensor unit is configured to transmit the indication when the presence of the electronic tag is no longer detected.

Clause 11. The fluid flow disconnect system of any one of Clauses 1-10, wherein at least the second sensor unit comprises a memory device for storing a patient identifier, the system further comprising: a non-transitory computer readable medium having instructions stored thereon that, when executed by a computing device, cause the computing device to perform operations comprising: associating, at the computing device, the patient identifier with the first and second sensor units; storing the patient identifier in the memory device; receiving the indication regarding whether the second sensor unit is within the threshold distance of the first sensor unit; obtaining, from at least the first sensor unit, the patient identifier in connection with receiving the indication regarding whether the first and second portions of the fluid connector assembly are coupled together responsive to the sensing; confirming whether the obtained patient identifier is associated with the first and second sensor units; and providing, for display based on the obtaining and confirming, a coupling status of the first and second portions of the fluid connector assembly and whether the obtained patient identifier is associated with the first and second sensor units.

Clause 12. The fluid flow disconnect system of any one of Clauses 1-11, wherein the first sensor unit comprises a piezo-electric circuit configured to, when activated, generate an audible alarm, and wherein when the threshold distance becomes no longer satisfied by way of the first and second portions no longer being coupled together, the first sensor unit is configured to cause the piezo-electric circuit to generate the audible alarm.

Clause 13. A disconnect sensor comprising: a first sensor unit configured to be removably coupled to a first portion of a fluid connector assembly; and a second sensor unit configured to be removably coupled to a second portion of the fluid connector assembly and configured to be detected by the first sensor unit, wherein the first sensor unit comprises a sensor configured to sense when the second sensor unit is within a threshold distance of the first sensor unit, and comprises a transmitter configured to transmit, to a device remote from the disconnect sensor, an indication regarding whether the first and second portions of the fluid connector assembly are coupled together responsive to the sensing; wherein the first and second sensor units are configured such that the threshold distance is satisfied when the first and second portions of the fluid connector assembly are coupled together to form the fluid connector assembly and the threshold distance is not satisfied when the first and second portions are not coupled together.

Clause 14. The disconnect sensor of Clause 13, wherein the first sensor unit is configured to be removably coupled to the first portion of the fluid connector assembly via one or more first tabs secured to an exterior the first portion of the fluid connector assembly, and the second sensor unit is configured to be removably coupled to the second portion of the fluid connector assembly via one or more second tabs secured to an exterior of the second portion of the fluid connector assembly.

Clause 15. The disconnect sensor of Clause 14, wherein the first and second sensor units each include a pair of parallel locking appendages that, when the respective sensor unit is coupled to a respective portion of the fluid connector assembly, straddle the respective portion of the fluid connector assembly on opposing sides of the respective portion fluid connector assembly and lock into respective key openings within the tabs of the respective portion of the fluid connector assembly on the opposing sides so that the sensor unit is held against a side of the respective portion of the fluid connector assembly between the tabs.

Clause 16. The disconnect sensor of Clause 13 or Clause 14, wherein at least the second sensor unit comprises a memory device for storing a patient identifier, and is further configured to: wirelessly receive the patient identifier from a computing device remote from the first and second sensor units; store the patient identifier in the memory device; and wirelessly transmit, to the computing device, the patient identifier and the indication regarding whether the first and second portions of the fluid connector assembly are coupled together responsive to the sensing.

Clause 17. A method, comprising: providing a first sensor unit and a second sensor unit, the first sensor unit configured to be removably coupled to a first portion of a fluid connector assembly, and the second sensor unit configured to be removably coupled to a second portion of the fluid connector assembly and configured to be detected by the first sensor unit; receiving an indication regarding whether the second sensor unit is within a threshold distance of the first sensor unit; and providing, for display based on receiving the indication, a coupling status of the first and second portions of the fluid connector assembly.

Clause 18. The method of Clause 17, further comprising: wirelessly receiving, from the first sensor unit, at a mobile computing device remote from the first and second sensor units, an indication that the second sensor unit is no longer within the threshold distance of the first sensor unit; and responsive to indication that the second sensor unit is no longer within the threshold distance of the first sensor unit, providing an alarm at the mobile computing device indicating that the first portion of the fluid connector assembly has become decoupled from the second portion of the fluid connector assembly.

Clause 19. The method of Clause 17 or Clause 18, further comprising: associating a patient identifier with a first sensor unit and a second sensor unit; obtaining, from at least the first sensor unit, the patient identifier in connection with receiving the indication regarding whether the first and second portions of the fluid connector assembly are coupled together responsive to the sensing; and confirming whether the obtained patient identifier is associated with the first and second sensor units before providing the coupling status; and providing an indication of whether the obtained patient identifier is associated with the first and second sensor units.

Clause 20. A non-transitory computer-readable memory having instructions stored thereon that, when executed, perform operations that facilitate the method of any one of Clauses 17-19.

### Further Considerations:

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation, or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™.NET™, C, C++, web services, or rich site summary (RSS). In some embodiments, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “correspond” or “corresponding” when used to describe a relationship between two or more elements encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

What is claimed is:

1. A fluid flow disconnect system comprising:
a first sensor unit configured to be removably coupled to a first portion of a fluid connector assembly; and
a second sensor unit configured to be removably coupled to a second portion of a fluid connector assembly and configured to be detected by the first sensor unit,
wherein the first sensor unit comprises a sensor configured to sense when the second sensor unit is within a threshold distance of the first sensor unit, and comprises a transmitter configured to transmit, to a device remote from the first and second sensor units, an indication regarding whether the first and second portions of the fluid connector assembly are coupled together responsive to the sensing; and
wherein the first and second sensor units are configured such that the threshold distance is satisfied when the first and second portions of the fluid connector assembly are coupled together to form the fluid connector assembly and the threshold distance is not satisfied when the first and second portions are not coupled together.

2. The fluid flow disconnect system of claim 1, wherein the first sensor unit is configured to be removably coupled to the first portion of the fluid connector assembly via one or more first tabs secured to an exterior the first portion of the fluid connector assembly, and the second sensor unit is configured to be removably coupled to the second portion of the fluid connector assembly via one or more second tabs secured to an exterior of the second portion of the fluid connector assembly.

3. The fluid flow disconnect system of claim 2, wherein the first and second sensor units each include a pair of parallel locking appendages that, when the respective sensor unit is coupled to a respective portion of the fluid connector assembly, straddle the respective portion of the fluid connector assembly on opposing sides of the respective portion of the fluid connector assembly and lock into respective key openings within the tabs of the respective portion of the fluid connector assembly on the opposing sides so that the sensor unit is held against a side of the respective portion of the fluid connector assembly between the tabs.

4. The fluid flow disconnect system of claim 3, wherein, when a respective sensor unit is coupled to a respective portion of the fluid connector assembly, a majority portion of the sensor unit having at least half of a mass of the sensor unit is held by the locking appendages in a position perpendicular to the side of the respective portion of the fluid connector assembly between the tabs, wherein the majority portion of the first sensor unit comprises at least one flat side facing a flat side of the majority portion of the second sensor unit when the sensor units are held by the locking appendages.

5. The fluid flow disconnect system of claim 4, wherein the majority portion of a respective sensor unit comprises an overhang portion that extends away from the respective sensor unit to align with and couple to an interface in the majority portion of the other sensor unit when the first and second sensor units are held by the locking appendages and the first and second portions of the fluid connector assembly are coupled together.

6. The fluid flow disconnect system of claim 5, wherein interface or the overhang portion comprises a trigger contact that is activated responsive to the overhang portion aligning with and coupling to the interface, and wherein the first sensor unit is configured to detect the second sensor unit when the trigger contact is activated and to detect that the first and second sensor units are disconnected with the trigger contact is deactivated.

7. The fluid flow disconnect system of claim 6, wherein the trigger contact comprises a push button or a contact for completing an electrical circuit.

8. The fluid flow disconnect system of claim 1, wherein the transmitter is configured to wirelessly transmit the indication to the remote device.

9. The fluid flow disconnect system of claim 1, further comprising the first portion of the fluid connector assembly and the second portion of the fluid connector assembly, wherein the first portion of the fluid connector assembly comprises a first portion of a valve and a first fluid passageway therein; and
wherein the second portion of the fluid connector assembly comprises a second portion of the valve and a second fluid passageway therein, the second portion of the fluid connector assembly being configured to become coupled to the first portion of the fluid connector assembly to form a continuous fluid passage from the first and second fluid passageways.

10. The fluid flow disconnect system of claim 1, wherein the second sensor unit comprises a electronic tag, and the sensor of the first sensor unit is configured to wirelessly detect a presence of the electronic tag of the second sensor unit while the first and second sensor units are brought together by way of the first and second portions of the fluid connector assembly being coupled together to form the fluid connector assembly, and configured to no longer detect the presence of the electronic tag when the first and second sensor units are moved away from each other by way of the first and second portions of the fluid connector assembly being decoupled, and wherein the first sensor unit is configured to transmit the indication when the presence of the electronic tag is no longer detected.

11. The fluid flow disconnect system of claim 1, wherein at least the second sensor unit comprises a memory device for storing a patient identifier, the system further comprising:
a non-transitory computer readable medium having instructions stored thereon that, when executed by a computing device, cause the computing device to perform operations comprising:
associating, at the computing device, the patient identifier with the first and second sensor units;
storing the patient identifier in the memory device;
receiving the indication regarding whether the second sensor unit is within the threshold distance of the first sensor unit;
obtaining, from at least the first sensor unit, the patient identifier in connection with receiving the indication regarding whether the first and second portions of the fluid connector assembly are coupled together responsive to the sensing;
confirming whether the obtained patient identifier is associated with the first and second sensor units; and
providing, for display based on the obtaining and confirming, a coupling status of the first and second portions of the fluid connector assembly and whether the obtained patient identifier is associated with the first and second sensor units.

12. The fluid flow disconnect system of claim 1, wherein the first sensor unit comprises a piezo-electric circuit configured to, when activated, generate an audible alarm, and wherein when the threshold distance becomes no longer satisfied by way of the first and second portions no longer being coupled together, the first sensor unit is configured to cause the piezo-electric circuit to generate the audible alarm.

13. A disconnect sensor comprising:
a first sensor unit configured to be removably coupled to a first portion of a fluid connector assembly; and
a second sensor unit configured to be removably coupled to a second portion of the fluid connector assembly and configured to be detected by the first sensor unit,
wherein the first sensor unit comprises a sensor configured to sense when the second sensor unit is within a threshold distance of the first sensor unit, and comprises a transmitter configured to transmit, to a device remote from the disconnect sensor, an indication regarding whether the first and second portions of the fluid connector assembly are coupled together responsive to the sensing;
wherein the first and second sensor units are configured such that the threshold distance is satisfied when the first and second portions of the fluid connector assembly are coupled together to form the fluid connector assembly and the threshold distance is not satisfied when the first and second portions are not coupled together.

14. The disconnect sensor of claim 13, wherein the first sensor unit is configured to be removably coupled to the first portion of the fluid connector assembly via one or more first tabs secured to an exterior the first portion of the fluid connector assembly, and the second sensor unit is configured to be removably coupled to the second portion of the fluid connector assembly via one or more second tabs secured to an exterior of the second portion of the fluid connector assembly.

15. The disconnect sensor of claim 14, wherein the first and second sensor units each include a pair of parallel locking appendages that, when the respective sensor unit is coupled to a respective portion of the fluid connector assembly, straddle the respective portion of the fluid connector assembly on opposing sides of the respective portion fluid connector assembly and lock into respective key openings within the tabs of the respective portion of the fluid connector assembly on the opposing sides so that the sensor unit is held against a side of the respective portion of the fluid connector assembly between the tabs.

16. The disconnect sensor of claim 13, wherein at least the second sensor unit comprises a memory device for storing a patient identifier, and is further configured to:
wirelessly receive the patient identifier from a computing device remote from the first and second sensor units;
store the patient identifier in the memory device; and
wirelessly transmit, to the computing device, the patient identifier and the indication regarding whether the first and second portions of the fluid connector assembly are coupled together responsive to the sensing.

17. A method, comprising:
providing a first sensor unit and a second sensor unit, the first sensor unit configured to be removably coupled to a first portion of a fluid connector assembly, and the second sensor unit configured to be removably coupled to a second portion of the fluid connector assembly and configured to be detected by the first sensor unit;
receiving an indication regarding whether the second sensor unit is within a threshold distance of the first sensor unit; and
providing, for display based on receiving the indication, a coupling status of the first and second portions of the fluid connector assembly.

18. The method of claim 17, further comprising:
wirelessly receiving, from the first sensor unit, at a mobile computing device remote from the first and second sensor units, an indication that the second sensor unit is no longer within the threshold distance of the first sensor unit; and
responsive to indication that the second sensor unit is no longer within the threshold distance of the first sensor unit, providing an alarm at the mobile computing device indicating that the first portion of the fluid connector assembly has become decoupled from the second portion of the fluid connector assembly.

19. The method of claim 17, further comprising:
associating a patient identifier with a first sensor unit and a second sensor unit;
obtaining, from at least the first sensor unit, the patient identifier in connection with receiving the indication regarding whether the first and second portions of the fluid connector assembly are coupled together responsive to the sensing; and
confirming whether the obtained patient identifier is associated with the first and second sensor units before providing the coupling status; and
providing an indication of whether the obtained patient identifier is associated with the first and second sensor units.

20. A non-transitory computer-readable memory having instructions stored thereon that, when executed, perform operations that facilitate the method of claim 17.
